(19) United States
(12) Patent Application Publication
(10) Pub. No.: US 2004/0171049 A1
(43) Pub. Date: Sep. 2, 2004

(54) Methods and probes relating to Smith-Magenis syndrome and the RAI1 gene

(76) Inventor: Sarah H. Elsea, Okemos, MI (US)

Correspondence Address: PRICE HENEVELD COOPER DEWITT & LITTON, LLP, 695 KENMOOR, S.E., P O BOX 2567, GRAND RAPIDS, MI, 49501, US

(21) Appl. No.: 10/746,834

(22) Filed: Dec. 24, 2003

Related U.S. Application Data
(60) Provisional application No. 60/436,437, filed on Dec. 24, 2002.
(60) Provisional application No. 60/449,649, filed on Feb. 24, 2003.

(51) Int. Cl. C12Q 1/68

(52) U.S. Cl. 435/6

(57) ABSTRACT

Method and probes are disclosed to assist in the diagnosis of Smith-Magenis syndrome (SMS). These methods include the use of probes that are specific for the retinoic acid induced (RAI1) gene. The probes are added to a genetic sample from a subject and the presence or sence of the RAI1 gene is determined. Alternatively, the genetic sample from the subject is sequenced to determine whether there is a mutation in the RAI1 gene. The deletion or mutation of the RAI1 gene leads to most of the phenotypic features of SMS.

This application claims the benefit of the filing date of U.S. Provisional Patent Application Serial No. 60/436,437, entitled “METHODS AND PROBES RELATING TO SMITH-MAGENIS SYNDROME AND THE RAI1 GENE,” by Sarah H. Elsea, filed Dec. 24, 2002; and U.S. Provisional Patent Application Serial No. 60/449,649, entitled “METHODS AND PROBES RELATING TO SMITH-MAGENIS SYNDROME AND THE RAI1 GENE, by Sarah H. Elsea, filed Feb. 24, 2003, the entire disclosures of which are hereby incorporated by reference.

### GOVERNMENT INTERESTS

The U.S. Government has a paid-up license in the invention and the right in limited circumstances to require the patent owner to license others on reasonable terms as provided by the terms of Grant No. HD38534A 01A2 awarded by the National Institute of Child Health and Human Development.

### BACKGROUND OF THE INVENTION

Smith-Magenis syndrome (SMS) is a multiple congenital anomalies and mental retardation syndrome that encompasses some unique characteristics, including unusual behavior abnormalities, sleep disturbance with inversion of the circadian rhythm of melatonin, distinct craniofacial and skeletal anomalies, moderate mental retardation, and significant speech delay. SMS patients have a recognizable physical phenotype that includes characteristic facies, brachycephaly, brachydactyly, hearing loss, myopia and hoarse voice. Though SMS is caused by a deletion or mutation of genetic material, it usually does not run in families in most cases. The deletion occurs due to an error in the sperm or egg and the parents are not “carriers” of SMS. The birth prevalence of SMS is estimated to be approximately 1:25,000, although SMS is likely under diagnosed due to the fact that it is a recently-described syndrome and its specific features (phenotype) can be subtle.

An individual with SMS may have just a few or many different clinical features. The clinical features include developmental delay, learning disability, mental retardation, low muscle tone in infancy, feeding problems in infancy, short stature, flat facial features, prominent jaw in older children and adults, abnormalities of the palate, with or without cleft lip, downturned mouth, unusually formed ears, chronic ear infections, hearing impairments, eye problems, including strabismus, and nearsightedness, short fingers and toes, heart defects and murmurs, urinary system problems, scoliosis, unusual gait, and sleep problems. While some individuals with SMS may not show significant behavior problems, almost always some degree of self injury and sleep disturbance occurs. Behavioral problems include: hyperactivity; head banging; hand biting; picking at skin, sores and nails; pulling off fingernails and toenails; explosive outbursts; tantrums; destructive and aggressive behavior; excitability; and arm hugging/hand squeezing when excited. Diagnosis of SMS is usually confirmed through a blood test called high resolution chromosome analysis which determines the karyotype or by fluorescence in situ hybridization (FISH).

In situ hybridization is the hybridization of a probe to a target. Hybrids are produced between the probe and the target as a result of an in situ hybridization procedure. FISH involves in situ hybridization with a fluorescent marker on the probe. Several definitions are relevant to the creation of probes for FISH.

The term “probe” refers to a polynucleotide, or mixture of polynucleotides, such as DNA sequence(s) or DNA segment(s), which has (or have) been chemically combined with individual label-containing moieties. Each such polynucleotide of a probe is typically single stranded at the time of hybridization to a target. For purposes of this application, the term “probe” will include “clones” as defined below.

The term “label” or “label-containing moiety” refers, in a general sense, to a moiety, such as a radioactive isotope or group containing the same, non-isotopic labels, and the like. Luminescent agents, depending upon the source of exciting energy, can be classified as radio luminescent, chemiluminescent, bioluminescent, and photoluminescent.

The term “linking compound” refers to a hydrocarbonaceous moiety with a linking compound with a nucleotide sequence. A linking compound is also capable of reacting with a fluorophore compound.

The term “clone” or equivalent refers to the process, wherein a particular nucleotide segment or sequence is inserted into an appropriate vector. The vector is then transported into a host cell, and the vector within the host is then caused to reproduce itself in a culturing process, thereby producing numerous copies of each vector and the respective nucleotide sequences that it carries. Cloning results from the formation of a colony of identical host cells, wherein each contains one or more copies of a vector incorporating a particular nucleotide segment or sequence. A nucleotide segment or sequence is now said to be “cloned” and the product nucleotide segments or sequences can be called “clones.”

Fluorescent markers for use in FISH are well known in the art. Fluorescent markers will produce light while being acted upon by radiant energy, such as ultraviolet lights or x-rays. Some of the probes that have been used for FISH have used fluorescent compounds that incorporate at least one fluorophore substituent (or group) per molecule and also one functional (i.e., reactive) substituent (or group) per molecule. Fluorescent compounds containing one to about three fluorophore substituents per fluorescent compound molecule have been used. A starting fluorescent compound has a molecular weight, which is not more than about 5000 and preferably not more than 1000, because larger molecular weights may possibly have an adverse effect upon the hybridization capacity of a product probe, with a complementary target sequence. Exemplary fluorescent compounds and linking compounds are well known and described in U.S. Pat. No. 5,663,319, for example.

The functional substituent is chosen so as to be reactive with a second functional substituent remaining incorporated into a linking group in a transaminated polynucleotide. In transanimation, a minor fraction of the total deoxycytidine bases that are contained in the starting specific chromosomal DNA sequences and segments become transaminated with an amino group of a difunctional linking compound (as defined above). The transanimation can be accomplished under aqueous liquid phase conditions in the presence of a bisulfate catalyst. The linking group is derived from a linking compound. For example, ratchet substituent can be chosen to be reactive with an amino substituent, or a carboxyl substituent, which is in the acid or salt form. Thus, the fluorescent labels are covalently linked to the probe DNA sequence.

For purposes of reactivity with such an amino substituent in a linking group, the reactive substituent of fluorescent compound has been of an amine-reactive functionality, such as a carboxyl substituent that is in the acide or salt form, an aldehyderadical or the like. The reactive substituents that have been used include those selected from, in an exemplified body, the group consisting of isothiocyanates, N-hydroxysuccinimide, esters, sulfonyl chlorides, carboxylic acid, azides, and the like.

For purposes of reactivity with such a carboxyl substituent in a linking group, the reactive substituent of the fluorescent compound has been of a carboxyl-reactive functionality, such as amino substituent, which is in a primary or secondary form or the like. The reactive substituents that have been used include a primary amino substituent, a thiol, a phosphate, ester, or the like.

The SMS critical interval was first described in 1996 and delineated in 1997 by using FISH and rodent: human somatic cell hybrid mapping experiments in patient samples harboring unusual or small deletions along chromosome 17. At that time, the SMS critical interval was reported to be approximately 1.5-2 Mb and located between cosmid cCI17-638 distally and the marker D17S29 proximally. Recently, the SMS critical region was further narrowed to approximately 950 kb, bordered distally by the PEMT gene and proximally by the FLII gene. Even though this region of chromosome 17 is extremely gene rich, until recently no single gene was reported to contribute to any of the major phenotypic characteristics seen in SMS. SMS was thought to be a contiguous gene syndrome, where multiple genes contributing to the syndrome phenotype are only related by their proximity to each other and not by function. In general, any fluorophore substituent or group can be employed as a starting fluorescent compound. However, because numerous genes are included in the deletion and because it has not previously been known which specific missing gene or genes is/are responsible for the phenotypic features of SMS, the existing clones or probes are hit or miss. They do not focus on the specific genes affected in this genetic syndrome.

Several SMS probes have been made commercially available. These probes include the Vysis SMS probe, Cytocell SMS probe, as well as Oncor D17S29 and D17S258 probes. However, using these probes was a bit like “shooting in the dark,” in that a) it was not known specifically which gene or genes in the chromosome 17 deletion was or were responsible for SMS, and b) it was not known whether or not these probes hybridized to that gene or those genes.

### SUMMARY OF THE INVENTION

In the present invention, it has been discovered that it is the deletion or mutation of the retinoic acid induced 1 (RAI1) gene which is responsible for most of the phenotypic features consistent with SMS.

The present invention comprises methods for diagnosing SMS by detecting whether the RAI1 gene has been deleted in a subject. Alternatively, the method involves doing a mutation sequence to determine whether the RAI1 gene has been mutated in a cellular sample of a given subject. The invention also comprises the identification of specific clones or probes capable of hybridization with the RAI1 gene such that detection of the presence of the RAI1 gene in a cellular sample is possible, and fashioning probes by attaching fluorescent tags thereto.

These and other features, advantages and objects of the present invention will be further understood and appreciated by those skilled in the art by reference to the following specification, claims and appended drawings.

### BRIEF DESCRIPTION OF THE DRAWINGS

FIG. 1 is an idiogram of chromosome 17 with a vertical indicator bar indicating the region that is deleted in SMS patients.

FIG. 2 shows the mapped location of the typical deletion at chromosome 17p11.2.

FIG. 3 illustrates a map of SMS probes.

FIG. 4 illustrates the results of FISH showing a deletion in chromosome 17p11.2.

FIG. 5 illustrates the results of FISH showing no deletion in chromosome 17p1 1.2.

### DETAILED DESCRIPTION OF THE PREFERRED EMBODIMENTS

Introduction

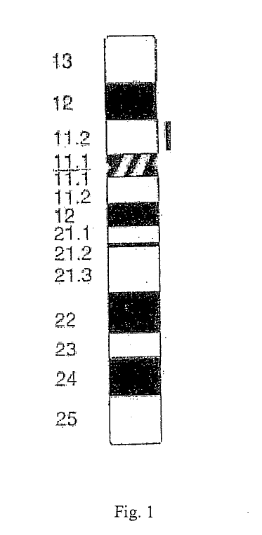

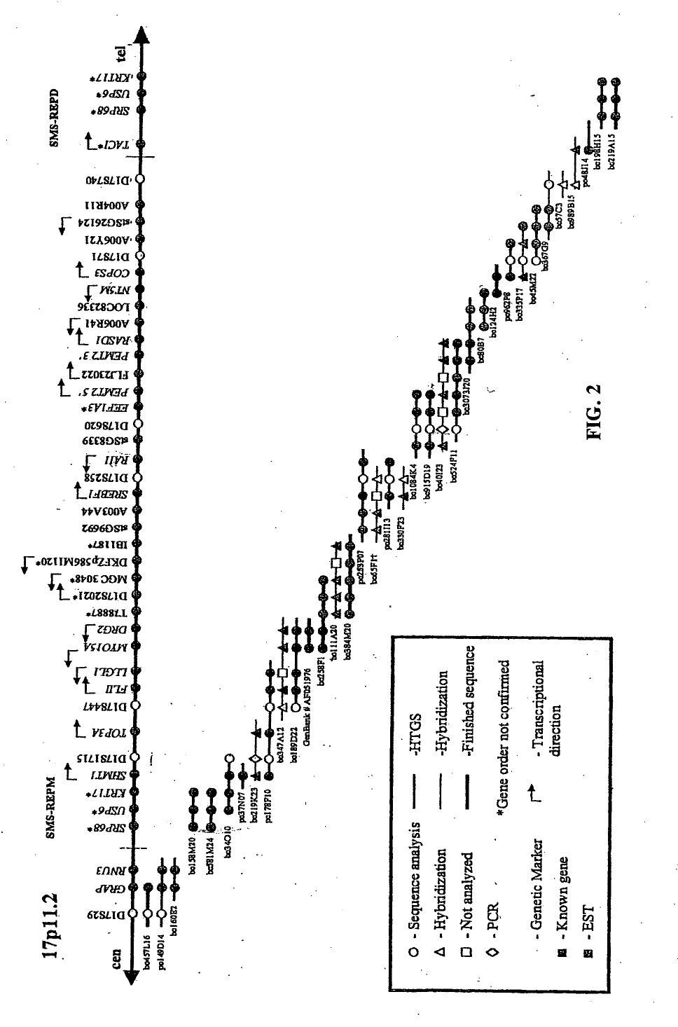

In order to identify mutations that may contribute to the SMS phenotype, the coding regions of several of the genes within the critical region of the RAI1 gene in patients that have been identified as having features that are consistent with SMS, yet who do not have a deletion at 17p11.2, as shown in FIGS. 1 and 2, were amplified and sequenced. RAI1 was identified as a mutated gene in the patients and is therefore linked to many of the phenotypic features of SMS. Thus, the absence (deletion) or mutation of RAI1 on one chromosome 17 homolog is diagnostic for SMS.

Detecting a Deletion

In the first preferred embodiment, the presence or absence of the RAI1 gene on one of the pair of chromosomes 17 is determined by conducting in situ hybridization of the RAI1 gene, if present, with one of several labeled clones. These clones either incorporate a suitable marker (label) or a binding site for a suitable marker (label) such that the presence of the RAI1 gene on a chromosome 17 can be detected. If the RAI1 gene has been heterozygously deleted, its presence will be identified on only one of the two chromosomes 17.

The method for detecting SMS comprises: obtaining a DNA sample of the subject; contacting the DNA sample with a nucleic acid probe capable of specifically hybridizing with the RAI1 gene, wherein the nucleic acid probe sequence is labeled with a detectable marker; and detecting whether the nucleic acid probe hybridized to the DNA sample.

The term “hybridize” refers to a method of interacting a nucleic acid probe with a DNA or RNA molecule. If a nucleic acid probe binds to the DNA or RNA molecule with high affinity, it is said to “hybridize” to the DNA or RNA molecule. The strength of the interaction between the probe and its target can be assessed by varying the stringency of the hybridization conditions. Various low or high stringency hybridization conditions may be used depending upon the specificity and selectivity desired. Stringency is controlled by varying salt or denaturant concentrations. Under stringent hybridization conditions, only highly complimentary nucleic acid sequences are hybridized. Preferably, such conditions prevent hybridization of the nucleic acids having one or two mismatches out of twenty contiguous nucleotides. Hybridization involves the binding of complementary strands of nucleic acid, for example, probe to target nucleic acid through hydrogen bonds which are similar to the bonds that would naturally occur in chromosomal DNA.

Useful polynucleotides for use in RAI1 probes include any DNA sequence or segment which can hybridize with a portion of the RAI1 gene. These useful polynucleotides include bacterial artificial chromosomes (BACs), P1 artificial chromosomes (PACs), and cosmids.

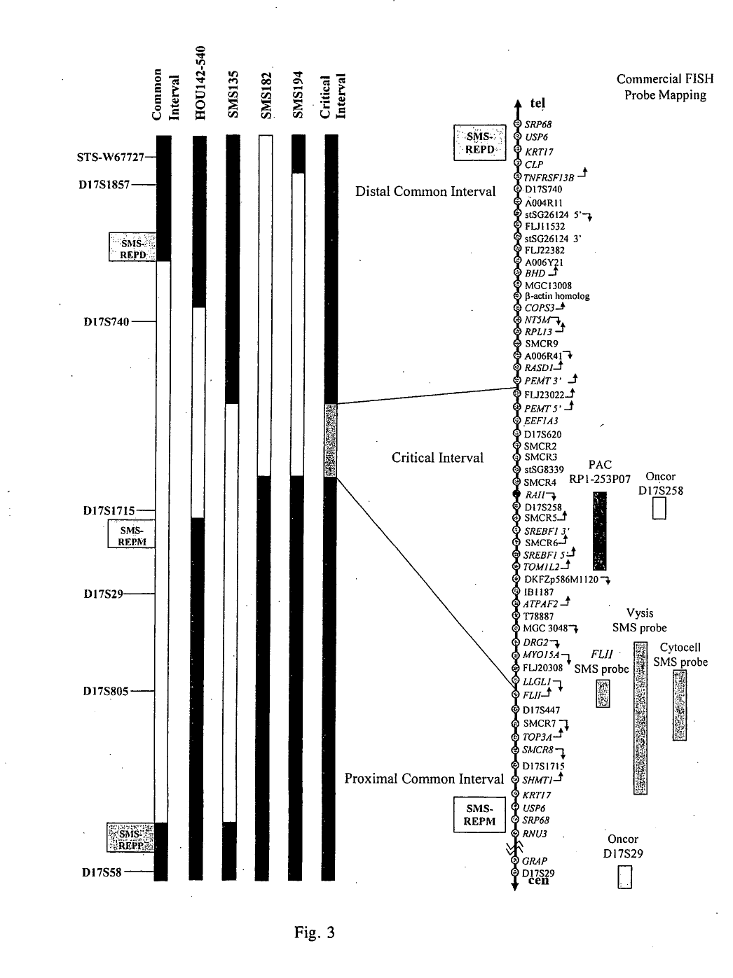

PAC clones for the RAI1 gene were identified from the RCPI-11 human PAC library. PACs were identified by hybridization using available STS markers and direct sequence analysis. The genomic clones which can detect the presence of RAI1 , include RPCI-1 253P07 and RPCI-1 281I13 which are publicly available clones, whose sequence are also publicly available at the U.C.S.C. and/or N.C.B.I. (See FIG. 3). Alternatively, flow-sorted chromosome 17 cosmids such as 83H6, 92C8, 94G3, 118C5, 128C5, 125B3 and 129D1 may be used as genomic probes. These can be obtained from, among other places, the Baylor College of Medicine. The following table also identifies RAI1 primers which can be used as the polynucleotide in an SMS probe for Southern blot analysis.

TABLE 1

| | | | | |
|---|---|---|---|---|
| RAI1 | | | Product | Annealing |
| Exon | Forward Primer | Reverse Primer | size | Temp. |

| | | | | |
|---|---|---|---|---|
| 1 | CCTTCCCTCCCTCCCTCCCTTCC | CACCCCTGCAGGTAGTGGCTG | 474 bp | 65° C. |
| 1 + 2 | CGCTATGCTGGTGAGGAGAGCC | CCGACTGGTAGGCATGAAGATTC | 484 bp | 64° C. |
| 2 | CCATGACAGGCCGCTGACTGC | CAGGGAGCTTGTCCTTCTGAAG | 533 bp | 62° C. |
| 2 + 3 | CTGACCACAGCCACTTCATGCC | CACGGACTCGGGCTTGGCCTTCG | 500 bp | 63° C. |
| 3 | CAGCTTCCTCTACTGCAACCAG | GCGAAGGCCACGGAAGGGTCTTC | 504 bp | 60° C. |
| 3 | GCCCGACTCCTTGCAGCTGGAC | CCGGTCAGCCTTGGCCACCTCGG | 508 bp | 65° C. |
| 3 | GGACTTCAAGCAGGAGGAGGTGG | CAGAGAGGCGTCCGAGGTGGTG | 493 bp | 64° C. |
| *3 | CACATGAAGCAGGTGAAGAGG | CTGGAGGCAGCCTTGGGTGAG | 482 bp | 65° C. |
| *3 | CGTTCTCTCACGGCCCTGAGTG | GCCACTGGCGTTGCTGCTGCTGC | 590 bp | 68° C. |
| *3 | GCGCTCAAAAGGAAGTCGGCCC | CCACATTTACCAGGCCTTCTTCC | 496 bp | 64° C. |
| *3 | CCCTTTCCGACAAAGACCGTGG | GTGTGGCCTGGCTGTTTCTGTG | 508 bp | 64° C. |
| *3 | TGGACTCTCCAAAGGCCCGCT | AGGCCCCAAGTGCATCGTGG | 600 bp | 60° C. |
| 4 | CCTGGCCACACTCCCTGGAGG | CTGCCGGAGCCTCCTTGCTGCAC | 497 bp | 64° C. |
| 5 | TGTGCAGCTGCCGCCACT | ACTCTGCAGATTGTCCCGAGA | 470 bp | 57° C. |
| 6 | GCACACACCACCAACCCTCACT | AATGCCTCATTTCCATGTCC | 450 bp | 62° C. |
| 7 | GCTTGAGGGCTGGGCTCCAAC | CAAAGGCCCAACCTCCAATACC | 501 bp | 64° C. |
| 8 | GGACTGTGAAGGAGGTGCGAGG | GGAGTGGAGTGGAGTGTGGAGG | 310 bp | 66° C. |
| 9 | GAGGCTCCTGTGCTACTTTGCC | GTTGACACAGCCCAACCATGTGC | 323 bp | 64° C. |
| 9 | GCACATGGTTGGGCTGTGTCAAC | GTCAATAAAGATACAACGATTG | 538 bp | 62° C. |
| 9 | CAGCTCGATACACACAATCTTC | CCGTTGTGCACCACCAGGGACC | 530 bp | 64° C. |
| 9 | GGTCCCTGGTGGTGCACAACGG | GTGGGAGACGGCTTTGTCCTGG | 543 bp | 64° C. |
| 9 | CCAGGACAAAGCCGTCTCCCAC | GACTGTGAAGTCCGAGGTCGTC | 420 bp | 57° C. |
| *Spl.v.1 | GAGTCCTCTGGCACCGAACGAG | GCCGCCTCTCGCAGCCACTCTG | 379 bp | 62° C. |
| Spl.v.2 | CTGCAGCCCCGGACTCC | TTGCAAGCGGCTGGCGAGAG | 302 bp | 62° C. |
| Spl.v.3 | CCCACACCACACAAAGCA | GCGCTCTTGCTCTCCTTCT | 502 bp | 59° C. |
| *Spl.v.4 | CAAATGTCACCCTCGCGTCC | GACCTGGGGAGCTCTGTAG | 236 bp | 62° C. |
| *Spl.v.5 | TGCTAGGCTGGTGGGAAAGG | CGGGATCTAGAAACTGGAAAGG | 282 bp | 62° C. |

RAI1 primers covering the primary transcript as well as splice variants. The annealing temperature for the primers is indicated. Exons in bold have identified mutations.
*Qiagen Q solution was routinely added to the PCR mix with these primer sets.

Southern blot analysis transfers denated DNA from agarose gels in which fragments have been separated by electrophoreses to a nitrocellulose or nylon membrane laid over the gel, before hybridization with a complementary nucleic acid probe. A buffer is drawn through the agarose gel by electroblotting or vacuum blotting procedures. Southern blotting analysis can thereby be used to identify a particular DNA sequence within a mixture of restriction fragments, for example, to determine the presence, position, and number of copies of a gene (RAI1).

The polynucleotides can be specific for the RAI1 gene, i.e., map only to the RAI1 gene or portion of the RAI1 gene. The polynucleotides can also map to the RAI1 gene or portion of the RAI1 gene and portions of other adjacent genes within p11.2 of chromosome 17. Such polynucleotides are considered nonspecific and include PAC RP1-253P07 or Oncor D17S258. (See FIG. 3).

Now that useful polynucleotides have been identified, these polynucleotides are formed into a probe which will include a fluorescent indicator.

As used herein, the term “label,” “marker,” or “indicating means” in their various grammatical forms refer to moieties that are either directly or indirectly involved in the production of the detectable signal. Any label or indicating means may be used that can be linked to the nucleic acid probes, including, without limitation, radioactive labels, enzymes, chromosomes and fluorogens. These labels may be used alone or in conjunction with additional reagents. Exemplary fluorescent compounds and methods for linking the compounds with a probe are described in U.S. Pat. No. 5,663,319.

A fluorescent label is preferred. The presence or absence of the RAI1 gene can be determined by FISH of one of the above labeled probes to the RAI1 gene. FISH probes are created using any of the above described polynucleotides by using nick translation to incorporate a fluorescent label, such as Spectrum Green or Spectrum Orange dUTP (Vysis, Inc.) by following manufacturer instructions. For example, probe DNA (100 ng PAC and 100 ng cosmid) was precipitated, hybridized to metaphase spreads and washed. The probe will recognize the RAI1 gene and physically bind to it through nucleotide pairing. The probe announces its presence through the label. The labeled RAI1 gene/probe product can be detected under a fluorescent microscope.

Preferably, a control is also used which is a labeled probe that is specific for an area of chromosome 17 which is not RAI1 or any other portion of p11.2. This probe, when used with the RAI1 probe, will show that two chromosomes 17 are present in the sample. This probe should be labeled in a similar manner as the RAI1 probe. Thus, presence of this label will confirm the presence of two chromosomes 17 to avoid obtaining a false positive resulting from inadvertent elimination of a second chromosome 17 from the cellular sample.

As discussed above, commercially available SMS probes exist (FIG. 3). Only one probe, the Oncor D17S258, which is no longer commercially available, maps to the RAI1 gene, and that association was only recently discovered. At the time the Oncor D17S258 probe was used, it was not known that this probe targeted the RAI1 gene, among other genes. It had never been used specifically to identify an RAI1 deletion. It has now been confirmed, based upon genomic sequence data, that this probe maps to a portion of the intron in the RAI1 gene. The remaining probes do not map to the RAI1 gene and, therefore, may provide a false negative result when used to detect SMS. A false negative result is obtained since the probes will not show the deletion of the RAI1 gene.

A method for creating an RAI1 gene probe and using the probe is shown in the following example:

### SPECIFIC EXAMPLE 1

RAI1 clones are used to create an SMS probe. The cells containing the RAI1 clones are gown in E. coli, the clone DNA is isolated and quantitated, and then the RAI1 clone DNA is labeled with a fluorescent label. The procedures for labeling followed the manufacturer's instructions (Vysis Nick-Translation Kit).

A cellular sample is taken from the patient. Chromosomes are prepared and denatured so that the two strands of the DNA double helix are separated for all pairs of chromosomes. The cellular sample can be denatured by enzymatic degradation and/or heating. Metaphase chromosomes are prepared for hybridization by incubating at 37° C. in 2×SSC for 30 minutes, dehydrated through an ethanol series, and allowed to dry.

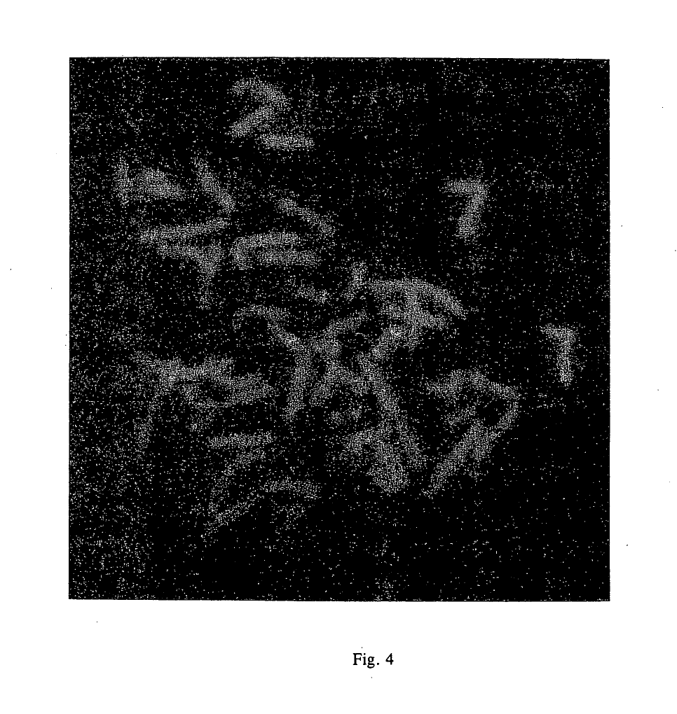

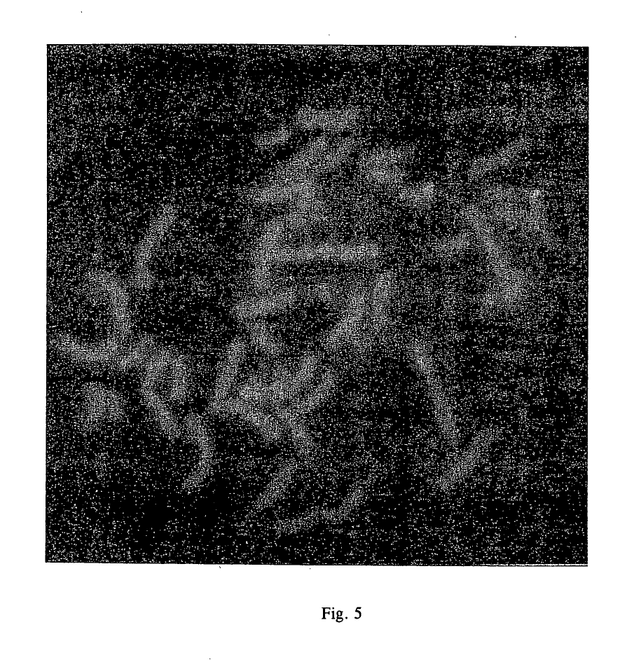

The labeled probe is then applied and hybridized to chromosomes for analysis. Hybridization and washing steps are carried out per manufacturer recommendations (Vysis, Inc.), then counterstained using Vectashield antifade with DAPI (Vector Labs). Analysis of the FISH experiments are carried out on a Zeiss Axioplan2 microscope and photographed with a Hamamatsu black and white camera using Zeiss Axio-Vision software version 2.0 (Carl Zeiss). Visualization of hybridization, utilizing a fluorescence microscope, can detect the presence of the two labels on each chromosome 17. If only one of the labeled chromosomes 17 contains the labeled RAI1 , then the patient has SMS. For example, FIG. 4 illustrates a patient that has SMS, and thus the deletion of 17p11.2, while FIG. 5 illustrates a patient that does not have a deletion at 17p11.2.

Detecting a Mutation

In another preferred embodiment, a method for detecting SMS in patients without a deleted RAI1 gene, involves determining whether there is a mutation in the RAI1 gene. This method involves obtaining a genetic sample from a subject and sequencing the sample to determine if there is a mutation in the RAI1 gene. The proper sequence for the RAI1 gene is publicly known. The presence of a mutation in the RAI1 gene will identify the subject as having SMS.

### SPECIFIC EXAMPLE 2

The sequencing reaction is performed by polymerase chain reaction (PCR) and the subsequent sequencing and analysis of PCR products. PCR primers covering the RAI1 coding sequence and alternative splice variants are listed in Table 1. PCR is performed in a 25 μL volume with a 50-200 ng template. PCR amplification is performed in an ABI thermocycler with the following conditions (unless otherwise noted in Table 1), initial denaturation at 94° C. for four minutes, 30 cycles of 94° for one minute, 64° C. for one minute, 72° C. for one minute, and a final extension of 72° C. for 10 minutes. PCR products of ˜500 base pairs are then electrophoresed in 2% TBE agarose gels containing ethidium bromide and gel purified using a commercially available Qiagen Gel Extraction Kit. A reaction containing at least 10-40 ng of PCR product template in distilled water and 30 pmol of sequencing primer are prepared. The PCR products are then sequenced, and sequences are compared to the known sequence of the RAI1 gene. The PCR primers can be used in SMS probes which detect a deletion of the RAI1 gene or for comparison to a cellular sample to determine if a mutation of the RAI1 gene has occurred.

Conclusion

The above description is considered that of the preferred embodiments only. Modification of the invention will occur to those skilled in the art and to those who make or use the invention. Therefore, it is understood that the embodiments shown in the drawings and described above are merely for illustrative purposes and not intended to limit the scope of the invention, which is defined by the following claims as interpreted according to the principles of patent law, including the doctrine of equivalents.

The invention claimed is:

1. A method of detecting the deletion or presence of the RAI1 gene, comprising: contacting a target nucleic acid with a reagent that detects heterozygous deletion of the RAI1 (retinoic acid induced 1) gene in a target nucleic acid.

2. The method of claim 1, wherein the reagent is selected from the group of cosmids 83H6, 92C8, 94G3, 118C5, 12533 and 129D1.

3. The method of claim 1, wherein the reagent is selected from the group of the RAI1 primers listed in Table 1.

4. The method of claim 1, wherein the reagent is a flow-sorted chromosome 17 cosmid that hybridizes to the RAI1 gene.

5. The method of claim 1, wherein the reagent is selected from the PACs consisting of RPCI-1 253P07; RPCI-281I13.

6. The method of claim 1, wherein the reagent is selected from the group consisting of: RPCI-1 253P07; RPCI-2 281I13; cosmids 83H6, 92C8, 94G3, 118C5, 12533 and 129D1; and the RAI1 primers listed in Table 1.

7. The method of claim 1, wherein fluorescent in situ hybridization is used to detect a hybridization of a fluorescently labeled probe.

8. The method of claim 1, further comprising the fluorescent labeling of the probe with a fluorescent label to indicate the presence of chromosome 17.

9. A method of detecting the deletion or presence of the RAI1 gene, comprising: applying a fluorescently labeled probe to the chromosomes of a patient, which will hybridize in the presence of the RAI1 gene, said probe including a reagent being selected from the group consisting of: RPCI-1 253P07; RPCI-2 281I13; cosmids 83H6, 92C8, 94G3, 118C5, 12533 and 129D 1; and the RAI1 primers listed in Table 1.

10. A method of diagnosing Smith-Magenis syndrome in a human subject comprising: conducting a sequence analysis of the subject chromosomes to determine whether a mutation of the RAI1 gene exists.

11. A method of diagnosing Smith-Magenis syndrome in a human subject comprising: contacting in situ the chromosome material of the subject with a fluorescently labeled reagent that will hybridize with the RAI1 gene in order to determine the presence of RAI1 on one or both of the chromosomes 17.

12. The method of claim 11, wherein said chromosomal material has been denatured prior to the in situ analysis.

13. The method of claim 11, wherein said chromosome material has been labeled.

14. A method of diagnosing Smith-Magenis syndrome in a human subject, comprising: contacting a target nucleic acid with a reagent that specifically detects heterozygous deletion of the RAI1 (retinoic acid induced 1) gene in a target nucleic acid.

15. The method of claim 14, wherein the reagent is selected from the group of cosmids 83H6, 92C8, 94G3, 118C5, 12533 and 129D1.

16. The method of claim 14, wherein the reagent is selected from the group of the RAI1 primers listed in Table 1.

17. The method of claim 14, wherein the reagent is a flow-sorted chromosome 17 cosmid that hybridizes to the RAI1 gene.

18. A probe for diagnosing Smith-Magenis syndrome comprising a reagent that detects the presence of the RAI1 gene and a fluorescent label that is attached to said reagent.

19. The probe of claim 18, wherein the reagent is RPCI-1 253P07.

20. The probe of claim 18, wherein the reagent is RPCI-1 281I13.

21. The probe of claim 18, wherein the reagent is a flow-sorted chromosome 17 cosmid that hybridizes to the RAI1 gene.

22. A probe for diagnosing Smith-Magenis syndrome comprising a reagent that detects the presence of the RAI1 gene.

23. The probe of claim 22, wherein the reagent is selected from the group consisting of the RAI1 primers listed in Table 1.
